(19) United States
(12) Patent Application Publication
(10) Pub. No.: US 2020/0246413 A1
(43) Pub. Date: Aug. 6, 2020

(54) COMPOSITION FOR TREATING FINE DUST-CAUSED SKIN CELL DAMAGE, COMPRISING PRUNUS MUME FLOWER EXTRACT

(71) Applicant: AMOREPACIFIC CORPORATION, Seoul (KR)

(72) Inventors: Hyoung-June KIM, Yongin-si (KR); Jinsup SHIM, Yongin-si (KR); Tae Ryong LEE, Yongin-si (KR)

(73) Assignee: AMOREPACIFIC CORPORATION, Seoul (KR)

(21) Appl. No.: 16/648,946

(22) PCT Filed: Aug. 13, 2018
(86) PCT No.: PCT/KR2018/009253

§ 371 Date: Mar. 19, 2020

(30) Foreign Application Priority Data
Sep. 21, 2017 (KR) 10-2017-0121801

(51) Int. Cl. A61K 36/736; A61K 8/9789; A61Q 19/00; A23L 33/105; B01D 11/02

(52) U.S. Cl. CPC A61K 36/736; A61K 8/9789; A23V 2002/00; A23L 33/105; B01D 11/0288; A61Q 19/00

(57) ABSTRACT

Disclosed in the present specification is a composition comprising a Prunus mume flower extract as an effective ingredient for treating fine dust-caused skin damage, wherein the expression of IL-36G (NM 019618), which is a gene having an expression level affected by fine dust in skin cells, is regulated to a novel level. By using the composition for treating fine dust-caused skin damage, gene expression changed by fine dust can return to a normal level to treat skin cell damage.

### BACKGROUND OF THE INVENTION

### Field of the Invention

The present disclosure discloses a composition for caring damages of skin cells by microdust, particularly a composition comprising Prunus mume flower extract and which takes care of damages of skin cells by significantly changing biomarkers, etc. which are skin cell genes whose expression levels are changed by microdust as compared with the normal state.

### Description of the Related Art

The skin is a part of the body that is directly exposed to the external environment. It not only serves as a protective layer for protecting important organs of our body, but also regulates moisture evaporation and protects the body from external infections. However, even though the skin is meant to prevent virus invasion from the outside, excessive exposure of the skin to ultraviolet rays or pollutants causes skin irritation. Particularly, the skin is damaged by Asian dust accompanied by strong wind and dirt.

Asian dust is a phenomenon in which small sands or red clays float from the inland deserts of China, Mongolia, etc., carried far away by the upper winds and then drops near the ground. In Korea, Asian dust occurs periodically every spring. Asian dust is a combination of organic and inorganic materials, and its physical characteristics and constituents are very diverse depending on the time and place of occurrence. It also includes metals that can give a biological effect. Large particles of Asian dust usually stay in their source or surroundings, and small particles thereof flow even into Korea. It is reported that when inhaled, this dust is deposited in the lower bronchial tubes and even in the gas exchange part of the lungs, which may cause damage to the respiratory system. In addition, it was found that damages of skin cells increased in the skin of people living in dusty areas or areas with a lot of Asian dust.

IL-36G is known as a useful biomarker in psoriasis caused by damages of skin cells by microdust. IL-36G is specific for psoriatic inflammation unlike the biomarkers such as 5100 proteins A7, A8, and A9, and IL-36G is known to be weakly expressed in other inflammatory skin diseases such as AD, CE, and LP (see Non-Patent Literature 2).

### CITATION LIST

### Non-Patent Literature

Non-Patent Literature 1: Kim, H. J., et al, “Transcriptome analysis of airborne PM2.5-induced detrimental effects on human keratinocytes”, Toxicology Letters 273, 26-35, 2017.
Non-Patent Literature 2: AM D'Erme et al, “IL-36c (IL-1F9) is a Biomarker for Psoriasis Skin Lesions”, Journal of Investigative Dermatology, Volume 135, 2015.

### SUMMARY OF THE INVENTION

The present inventors have found that microdust has harmful effects on the skin, which in turn affects the expression of skin cell genes, causing symptoms such as damages of skin cells.

Thus, one aspect of the present invention is to provide a composition for caring damages of skin cells by microdust.

In order to achieve the above object, one aspect of the present invention provides a composition for caring skin damages by microdust, comprising Prunus mume flower extract as an active ingredient, and which regulates the expression level of IL-36G (NM 019618), which is a gene in the skin cells whose expression levels are affected by microdust, to the normal level.

### BRIEF DESCRIPTION OF THE DRAWINGS

FIG. 1 shows the cell viabilities when treated with microdust extract. Here, ADSP, which represents Asian dust storm particle, refers to Asian dust, PM10 (particulate matter 10) refers to microdust having a particle size of 10 μm, and PM2.5 (particulate matter 2.5) refers to microdust having a particle size of 2.5μ, and the values on the x-axis of the graph represent the concentrations (μg/ml) of the microdust water-soluble extract of 0 μg/ml, 6.3 μg/ml, 12.5 μg/ml, 25 μg/ml, 50 μg/ml, 100 μg/ml and 200 μg/ml, respectively;

FIG. 2 shows that the mRNA expression level of IL-36G gene increases in skin cells irritated by PM2.5 microdust and returns to the normal level by treatment with Prunus mume flower extract.

### DETAILED DESCRIPTION OF THE PREFERRED EMBODIMENTS

Hereinafter, the present invention will be described in detail.

Prunus mume is a deciduous tree classified in the rosaceae rosales, which is originated from China and distributed in Japan, China, and Korea. The flower is called maewha in Korean, which is translated as plum blossom, and the fruit is called maesil in Korean, which is translated as plum. It is 5 to 10 m tall, the bark exhibits various colors such as yellowish white, greenish white, and red, and small branches may or may not have fine hairs. In the midland of Korea, the Primus mume flowers bloom earlier than the leaves in April, have colors in varying shades of white and red, and have a fragrant scent. The flowers have 5 calyxes with a round shape and several pieces of petals with a broad egg shape upside down. The leaves are alternate phyllotaxis, egg-shaped or broad egg-shaped, and 4 to 10 cm long. The leaves have sharp sawtooth on the edge, are hairy on both sides, and there are lines on the petiole. The plum of fruit is a ball-shaped drupe, is green, ripens in July to turn yellow with a diameter of 2 to 3 cm, has dense hairs, tastes sour, and the pulp hardly separates from the pit.

There are a great number of cultivars depending on the intended use, flower color, and petal shape. It is classified into Prunus mume for flower and Prunus mume for fruit depending on the intended use, and the Prunus mume for flower is again classified into a wild (yabai) type close to wild plant, a red (hibai) type which is a cultivar originated from a wild cultivar and has red flowers, and a bungo type which is a hybrid with apricot. Depending on the color of petals, it is classified into white Prunus mume with a white flower and a red Prunus mume with a red flower, and among the white Prunus mume, the white Prunus mume with white petals but green calyxes is classified into a green calyx form. Depending on the shape of the petals, those having only five pieces of petals are classified into a single flowered form and those having petals inside five pieces of petals are classified into a double flowered form.

In one aspect of the present invention, the composition for caring skin damages by microdust may comprise Prunus mume flower extract as an active ingredient.

In one aspect, the Prunus mume flower may be dried and pulverized.

In one aspect of the present invention, the Prunus mume flower may be extracted with a specific extraction solvent to form Prunus mume flower extract.

In one aspect of the present invention, the Prunus mume flower extract may be prepared by extracting Prunus mume flower with water or an organic solvent. Specifically, it may be prepared by extracting Prunus mume flower with at least one extraction solvent selected from the group consisting of water, C₁-C₆ anhydrous or hydrated lower alcohol, acetone, butylene glycol, ethyl acetate, diethyl acetate, diethyl ether, benzene, chloroform and hexane.

In one aspect, the Prunus mume flower extract may be extracted at room temperature.

In one aspect, the Prunus mume flower extract may be obtained by extracting with the extraction solvent and then further performing at least one of the following steps: filtration, concentration, separation or drying. In particular, the Prunus mume flower extract may be subjected to at least one filtration step.

In one embodiment, it is subjected to two filtration steps.

In one embodiment, the separation step may include a centrifugation step.

Specifically, the extraction may use at least one of: polar solvents including water, a C₁-C₆ anhydrous or hydrated lower alcohol, acetone and butylene glycol and low-polarity solvents including ethyl acetate, diethyl acetate, diethyl ether, benzene, chloroform and hexane.

More specifically, the solvent may be a 50-90% ethanol aqueous solution and may be a 60-80% or 65-75% ethanol aqueous solution. When the solvent is a 50-90% ethanol aqueous solution, the active ingredient can be effectively extracted from the Prunus mume flower. In one embodiment, the solvent may be an about 70% ethanol aqueous solution.

In one aspect, the extract may be concentrated under reduced pressure at an appropriate temperature in a distillation apparatus equipped with a cooling condenser after extraction.

However, the Prunus mume flower extract according to the present invention can be obtained by extraction according to a conventional method in the art, and the extraction method is not limited to the above-described methods.

In one aspect of the present invention, the composition may comprise 0.000001 to 30% by weight of Prunus mume flower extract based on the total weight of the composition. When the content thereof is 0.000001 to 30% by weight, the Prunus mume flower extract obtained an excellent effect of caring skin damages by microdust.

Specifically, the content may be 0.0000001% by weight or more, 0.0000005% by weight or more, 0.0000007% by weight or more, 0.0000009% by weight or more, 0.000001% by weight or more, 0.000002% by weight or more, 0.000004% by weight or more, 0.000006% by weight or more, 0.000008% by weight or more, 0.00001% by weight or more, 0.00003% by weight or more, 0.00005% by weight or more, 0.00007% by weight or more, 0.00009% by weight or more, 0.0001% by weight or more, 0.0003% by weight or more, 0.0005% by weight or more, 0.0007% by weight or more, 0.0009% by weight or more, 0.001% by weight or more, 0.01% by weight or more, 0.1% by weight or more, 1% by weight or more, 3% by weight or more, 5% by weight or more, 7% by weight or more, 9% by weight or more, 10% by weight or more, 13% by weight or more, 15% by weight or more, 17% by weight or more, 19% by weight or more, 21% by weight or more, 23% by weight or more, 25% by weight or more, 27% by weight or more, 29% by weight or more, 30% by weight or more, or 31% by weight or more and 32% by weight or less, 31% by weight or less, 30% by weight or less, 29% by weight or less, 28% by weight or less, 26% by weight or less, 24% by weight or less, 22% by weight or less, 20% by weight or less, 18% by weight or less, 16% by weight or less, 14% by weight or less, 12% by weight or less, 10% by weight or less, 9% by weight or less, 8% by weight or less, 6% by weight or less, 4% by weight or less, 2% by weight or less, 1% by weight or less, 0.1% by weight or less, 0.09% by weight or less, 0.04% by weight or less, 0.01% by weight or less, 0.006% by weight or less, 0.001% by weight or less, 0.0009% by weight or less, 0.0007% by weight or less, 0.00005% by weight or less, 0.00003% by weight or less, 0.00001% by weight or less, 0.000009% by weight or less, 0.000007% by weight or less, 0.000005% by weight or less, 0.000003% by weight or less, 0.000001% by weight or less, 0.0000009% by weight or less, 0.0000007% by weight or less, 0.0000005% by weight or less, or 0.0000003% by weight or less, 0.0000002% by weight or less, 0.0000001% by weight or less, 0.00000009% by weight or less, although not limited thereto.

Another aspect of the present invention includes the use of the composition for caring skin damages by microdust.

Another aspect of the present invention provides a method for caring skin damages by microdust in a subject, comprising administering an effective amount of Prunus mume flower extract to a subject in need thereof.

Another aspect of the present invention provides the use of Prunus mume flower extract for the preparation of compositions for caring skin damages by microdust.

Another aspect of the present invention provides Prunus mume flower extract for caring skin damages by microdust.

As used herein, the term “microdust” refers to a very small material that is invisible to the naked eye. It is a particulate matter that floats or flutters in the air for a long time and has a diameter of 10 μm or less. Especially, a particulate matter having a diameter of 2.5 μm or less is called “ultrafine particles”. In the present invention, “microdust” is intended to include “ultrafine particles”.

As used herein, the term “care” refers to effective protection of skin cells from irritation and inhibition, prevention or restoration (recovery) of a change of the expression level of a specific gene by the irritation.

In one aspect, the present invention provides a composition for inhibiting damages of skin cells by microdust by regulating the expression level of a specific gene in skin cells damaged by microdust to the normal level.

Specifically, in one aspect of the present invention, the genes in skin cells whose expression levels are affected by microdust include IL-36G (NM 019618). IL-36G (NM 019618) is a gene whose expression level is increased by microdust. Thus, it is possible to inhibit damages of skin cells by suppressing the expression level of this gene to the normal level (see Non-Patent Literature 1).

The gene used in the present invention and whose expression level is increased by microdust is shown in Table 1. In the table, the term “name” denotes genebank accession ID of NCBI, the term “gene symbol” denotes official gene symbol, and the term “gene title” denotes the name of each gene. These are described in Non-Patent Literature 1.

TABLE 1
Increased gene

| Name | Gene Symbol | Gene title |
|---|---|---|
| NM_019618 | IL36G | Interleukin 36, gamma |

The expression levels of the genes or proteins may be analyzed using various analysis methods known in the art, such as microarray, PCR, NGS (next generation sequencing), western blot, northern blot, ELISA, radioimmunoassay, radioimmunodiffusion, immunohistochemical staining, and immunoprecipitation assay.

Microdust causes damage to skin cells, which leads to inflammation and thus further damage to skin cells. The skin condition can be improved by taking care of the vicious cycle of the damages of skin cells with Prunus mume flower extract.

In one aspect of the present invention, the composition may be a cosmetic composition, a pharmaceutical composition, or a health functional food composition.

Examples of the cosmetic composition include cosmetics such as various creams, lotions, and toners and cleansing products, cleansers, soaps, and cosmetic solutions.

In one aspect, the cosmetics comprising the composition containing the Prunus mume flower extract of the present invention may be in the form of a solution, an emulsion, a viscous mixture or the like.

That is, in one aspect, the formulation of the cosmetics of the present invention is not particularly limited and may be, for example, an emulsion, a cream, a toner, an essence, a pack, a gel, a powder, a makeup base, a foundation, a lotion, an ointment, a patch, a cosmetic solution, a cleansing foam, a cleansing cream, a cleansing water, a body lotion, a body cream, a body oil, a body essence, a shampoo, a rinse, a body cleanser, a soap, a hair dye, or a spray.

Ingredients other than the Prunus mume flower extract may be selected and added to the cosmetic composition of each formulation without difficulty by those skilled in the art according to the desired formulation or purpose.

In addition, in one aspect, the cosmetics of the present invention may comprise a composition selected from the group consisting of water-soluble vitamins, oil-soluble vitamins, polypeptides, polysaccharides, sphingolipids and seaweed extracts.

In one aspect, the cosmetics of the present invention may contain ingredients that are generally used in cosmetics in addition to the essential ingredient, if necessary.

Examples of the additional ingredients include oil and fat ingredients, moisturizers, emollients, surfactants, organic and inorganic pigments, organic powders, UV absorbers, preservatives, sterilizers, antioxidants, plant extracts, pH adjusters, alcohols, colorants, fragrance, blood circulation stimulants, skin coolers, antiperspirants, and purified water.

However, the ingredients that may be contained in the cosmetics are not limited thereto. Also, the amount of any of the ingredients may be determined within a range not negatively affecting the purpose and effect of the present invention.

In one aspect, the pharmaceutical composition comprising the Prunus mume flower extract of the present invention may further comprise a suitable carrier, excipient and diluent conventionally used for preparation of pharmaceutical compositions.

The pharmaceutical composition comprising the Prunus mume flower extract may be formulated into any form suitable for pharmaceutical preparations, including oral formulations such as tablets, capsules, powders or syrups and agents for external application to the skin such as ointments, gels, creams, patches and sprays, according to a conventional method.

In general, it is to be understood that the actual dosage of the active ingredient administered by the pharmaceutical composition should be determined in light of various relevant factors such as the severity of the symptoms, the administration route, the age, gender, body weight and health condition of the subject. In general, the dosage of the active ingredient may be 0.0001 mg/kg/day to 3000 mg/kg/day, for example, 10 mg/kg/day to 500 mg/kg/day.

In the health functional food composition according to one aspect of the present invention, the health food may refer to a food manufactured from a nutrient which is likely to be deficient in normal diets or a raw material or an ingredient (functional raw material) with a function useful for the human body, and which maintains and improves health by maintaining the normal function of the human body or activating the physiological functions, although not limited thereto. The health food may be manufactured and processed into the form of a tablet, a capsule, a powder, a granule, a liquid, a pill, etc. However, the formulation is not limited thereto, and it may be manufactured and processed into any form under the law.

Specifically, a health beverage composition has no particular limitation on ingredients other than the above compound contained in the predetermined ratio as an essential ingredient. It may contain various flavoring agents or natural carbohydrates as additional ingredients as in conventional beverages. Examples of natural carbohydrates are conventional sugars such as monosaccharide, polysaccharide and cyclodextrin and sugar alcohols such as xylitol, sorbitol, and erythritol. Also, natural flavoring agents (thaumatin, stevia extract (e.g., rebaudioside A, glycyrrhizin, etc.)) and synthetic flavoring agents (e.g., saccharin, aspartame, etc.) may be used as the flavoring agent.

In general, it is to be understood that the actual dosage of the active ingredient administered by the health functional food composition should be determined in light of various relevant factors such as the severity of the symptoms, the administration route, the age, gender, body weight and health condition of the subject. In general, the dosage of the active ingredient may be 0.0001 mg/kg/day to 1000 mg/kg/day, for example, 0.02 mg/kg/day to 6 mg/kg/day.

Hereinafter, the constitution and effects of the present invention will be described in more detail with reference to examples. However, the following examples are provided for illustrative purposes only to facilitate understanding of the present invention, and the scope of the present invention is not limited thereto.

### Example 1: Preparation of Prunus mume Flower Extract

The Prunus mume flower was extracted at room temperature using an extraction solvent obtained by mixing purified water and ethanol at a ratio of 3:7, that is, 70% ethanol, as the extraction solvent. After extraction at room temperature, primary filtration was performed to remove the solid material contained in the extract. Then, the extract was concentrated to remove ethanol, followed by separation and purification. Then, the resultant was subjected to centrifugation and secondary filtration, and then dried to obtain Prunus mume flower extract.

### Example 2: Microdust Collection and Extraction

Microdust was collected using a low volume air sampler (Sensidyne, Gillian, Fla., U.S.A.). The filter and denuder of the filter pack were replaced at around 10:00 am on each measurement day to collect a sample for about 24 hours. Microdust was collected daily for 28 days in the downwind area of Seoul (Hankuk University of Foreign Studies, Center for International Studies, Residence Hall, 6th floor rooftop, Cheoin-gu, Yongin-si, Gyeonggi-do). The measurement time was measured by starting a timer when turning on a vacuum pump and recording the time of the timer when turning off the vacuum pump. The collection flow rate was set at 16.7 L/min. The flow rate was measured with a flowmeter (Model 4143, TSI Inc.) at the start of the measurement and measured again at the end of the measurement. The Teflon filter in the filter pack was weighed before and after collection. The Teflon filter was dried to a constant weight in a desiccator (NIKKO, Japan) at a relative humidity of 40% for 24 hours before measuring the weight of the Teflon filter, and then weighed twice on a five decimal place electronic balance (DVG215CD, Ohaus). The mean value was recorded. After the sample was collected, the Teflon filter was again dried to a constant weight in a desiccator for 24 hours before measuring the weight, and then weighed twice. Then, the mass concentration was calculated by comparing the measured weight with the weight measured before collection. Microdust was extracted by wetting the Teflon filter with 1 mL of ethanol and putting 14 mL of DW so that the aerosol collector surface of the filter reached the water surface, closing the lid and then applying ultrasonic waves to the filter with an ultrasonic cleaner for 30 minutes. In order to minimize the error caused by moisture in the filtration step, moisture was completely removed from the filter for 48 hours in a desiccator. Then, the weight of the filter was measured using an ultra-precision scale that can measure down to 0.1 mg (Mettler Toledo Company) to measure the weights of the filter before and after extraction.

### Example 3: Culture of Keratinocyte Lines (Normal Human)

Keratinocyte lines (normal human) were purchased from Lonza Inc. (Walkersville, Md., USA), subcultured, and then cultured in a CO2 incubator under the conditions of 37° C. and 5% CO2. The cell culture was performed in accordance with Lonza's guidelines. A KGM-2 bullet kit CC-4152 (ingredient: BPE (bovine pituitary extract)), human epidermal growth factor (hEGF), insulin, hydrocortisone, transferrin, epinephrine, and a KGM-2 bullet kit CC-3107 added with GA-1000 (gentamycin suflate+amphofericin-B) were added to 500 ml of KBM-2 CC-3103 medium to prepare the medium used in the cell culture.

### Example 4: Treatment of Keratinocyte Lines (Normal Human) with Microdust and Measurement of Cytotoxicity

In order to investigate cytotoxicity of treatment with microdust, MTT assay was performed with keratinocyte lines (normal human) according to the method of Mossman et al. (J. Immunol. Methods, 65, 55-63, 1983).

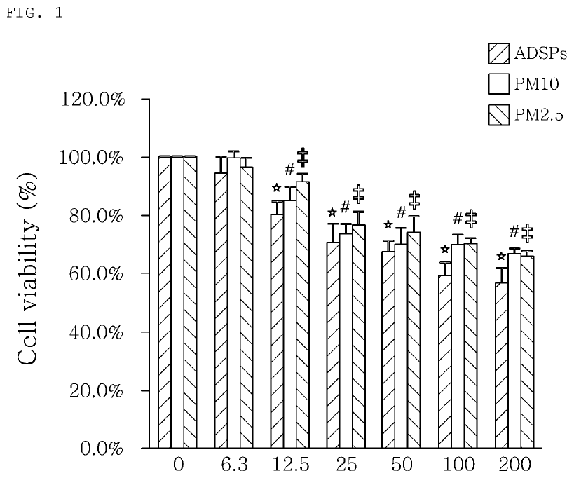

Specifically, a 24-well plate was used. The microdust with a diameter of 2.5 μm obtained in Example 3 was dispersed in purified water to prepare a microdust dispersion. Then, the microdust dispersion was applied to cells cultured under the conditions of Example 4 and 2.5×105 cells per well, followed by culture for 24 hours. Then, the cells were mixed with 5 mg/ml of MTT (3-4,5-dimethylthiazol-2,5-diphenyltetrazolium bromide) and further cultured at 37° C. for 3 hours. The medium was then removed and the formazan crystal formed was dissolved in 500 μl of DMSO. The lysate was aliquoted in a 96-well plate and the OD value was measured at 540 nm. The measurement results are shown in FIG. 1.

As shown in FIG. 1, the concentration (IC20) achieving an 80% survival rate in the cell line for cytotoxicity caused by a dispersion obtained by dispersing microdust of 2.5 micrometer or less was 12.5 μg/ml.

### Example 5: Investigation of Cell Gene Changes by Microdust Through Next Generation Sequencing

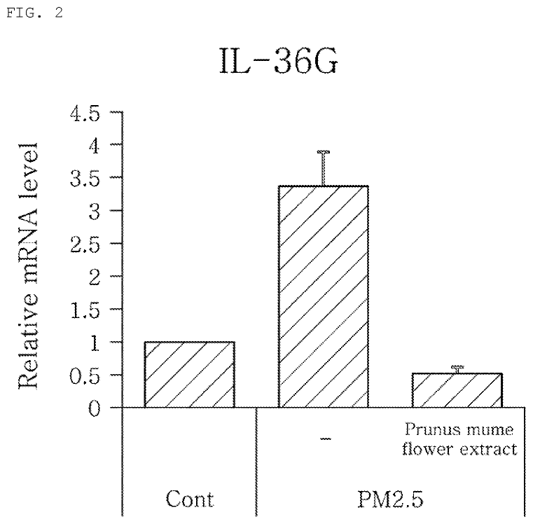

In order to carry out RNA-base sequence data processing and analysis, reference was made to the general analysis step developed by Trapnell et al. (2012). The RNA-seq data quality was determined using FastQC (http://www.bioinformatics.babraham.ac.uk/projects/fastqc/). The base and adapter sequences with low accuracy were removed using FASTX (http://hannonlab.cshl.edu/fastx_toolkit/). Then, alignment was performed using Tophat (Trapnell et al., 2009) and a human genome (hg19), and the amount of data of each sample was determined using EVER-seq renamed to RSeQC (Wang et al., 2012). In addition, the expression level of transcripts was quantified using Cufflinks, and transcription levels were compared between the sample treated with a microdust dispersion and a normal sample (Trapnell et al., 2010). A stringent cut-off of ≥2.0 fold-change, with FDR adjusted p-value <0.05, was used to determine the gene that showed a significant expression difference when treated with a dispersion of microdust with a diameter of 2.5 μm. The measurement results are shown in the following Table 2 and FIG. 2.

TABLE 2
Increased gene

| Name | Gene Symbol | Fold change |
|---|---|---|
| NM_019618 | IL36G | 7.1 |

### Example 6: Real-Time RT-PCR Quantification

Normal human keratinocytes cultured in Example 3 was treated with the microdust having a diameter of 2.5 μm extracted in Example 2 in the amount of 12.5 μg per 1 ml of the cell culture medium. Then, the relative mRNA expression level was measured using the primers (applied biosystems TaqMan® Primers) of genes indicated in Table 3 below. The extract prepared in Example 1 was used as the Prunus mume flower extract.

TABLE 3
Increased gene

| Name | Gene Symbol | TaqMan ® primer |
|---|---|---|
| NM_019618 | IL36G | Hs00219742_m1 |

The medium was treated with 20 ppm of Prunus mume flower extract. After 24 hours, the culture solution was removed and the cells were washed with 2 ml of phosphate buffered saline (PBS). Then, RNA was separated from the cells using Trizol reagent (Invitrogen, Carlsbad, Calif., USA). The separated RNA was further purified with an RNA kit (QIAGEN RNeasy kt, QIAGEN, Valencia, Calif.). Then, the quality of the RNA was determined using an Agilent 2100 BioAnalyzer (Agilent Technologies, Santa Clara, Calif., USA). cDNA was synthesized from the RNA using a reverse transcription kit (Superscript Reverse Transcriptase (RT) kit, Invitrogen, Carlsbad, Calif.). The cDNA was quantitatively analyzed by a real time-reverse transcription polymerase chain reaction (Q-RT-PCR) using the primers shown in Table 3. The changes of cell genes were evaluated by real-time PCR using TaqMan gene expression assay kit (Applied Biosystems, Foster City, Calif.). The results are shown in FIG. 2. Both of the Q-RT-PCR and the real-time PCR were performed according to the standard protocols distributed by Life Technologies. Specifically, 40 cycles of 95° C. for 20 seconds, 95° C. for 3 seconds and 60° C. for 30 seconds were performed.

FIG. 2 shows that there exist a gene whose expression level increases or decreases in skin cells irritated by microdust and that the expressions level of interleukin 26 gamma (IL-36G) was decreased by treatment with Prunus mume flower extract.

Therefore, it was found that Prunus mume flower extract effectively protect skin cells from irritation due to microdust and inhibits or prevents the expression levels of the above-described specific genes from changing due to the irritation, thereby achieving the normal expression levels.

Hereinafter, formulation examples of the compositions according to the present invention will be described. However, the cosmetic composition, pharmaceutical composition and health functional food composition may be formulated into various other forms. These examples are for illustrative purposes only and are not intended to limit the scope of the present invention.

### Formulation Example 1: Tablet

100 mg of the Prunus mume flower extract according to an embodiment of the present invention, 400 mg of lactose, 400 mg of corn starch, and 2 mg of magnesium stearate were mixed and subjected to a tableting process according to a conventional method for preparing tablets to prepare tablets.

TABLE 4

| Ingredients | Content (mg) |
|---|---|
| Prunus mume flower extract | 100 |
| Lactose | 400 |
| Corn starch | 400 |
| Magnesium stearate | 2 |

### Formulation Example 2: Capsule

100 mg of the Prunus mume flower extract according to an embodiment of the present invention, 400 mg of lactose, 400 mg of corn starch, and 2 mg of magnesium stearate were mixed and filled in a gelatin capsule according to a conventional method for preparing capsules to prepare a capsule.

TABLE 5

| Ingredients | Content (mg) |
|---|---|
| Prunus mume flower extract | 100 |
| Lactose | 400 |
| Corn starch | 400 |
| Magnesium stearate | 2 |

### Formulation Example 3: Granule

50 mg of the Prunus mume flower extract according to an embodiment of the present invention, 250 mg of anhydrous crystalline glucose, and 550 mg of starch were mixed and formulated into granules using a fluidized bed granulator. The granules were then filled in a pouch.

TABLE 6

| Ingredients | Content (mg) |
|---|---|
| Prunus mume flower extract | 50 |
| Anhydrous crystalline glucose | 250 |
| Starch | 550 |

### Formulation Example 4: Soap

TABLE 7

| Ingredients | Content (%) |
|---|---|
| Prunus mume flower extract | 5.00 |
| Oil and fat | q.s. |
| Sodium hydroxide | q.s. |
| Sodium chloride | q.s. |
| Fragrance | q.s. |
| Purified water | Balance |

### Formulation Example 5: Lotion

TABLE 8

| Ingredients | Content (%) |
|---|---|
| Prunus mume flower extract | 5.00 |
| L-ascorbic acid-2-phosphate | 1.00 |
| magnesium salt | |
| Water-soluble collagen (1% | 1.00 |
| aqueous solution) | |
| Sodium citrate | 0.10 |
| Citric acid | 0.05 |
| Licorice extract | 0.20 |
| 1,3-butylene glycol | 3.00 |
| Purified water | Balance |

### Formulation Example 6: Cream

TABLE 9

| Ingredients | Content (%) |
|---|---|
| Prunus mume flower extract | 3.00 |
| Polyethylene glycol monostearate | 2.00 |
| Self-emulsifying glycerin | 5.00 |
| monostearate | |
| Cetyl alcohol | 4.00 |
| Squalene | 6.00 |
| Glyceryl tri(2-ethylhexanoate) | 6.00 |
| Sphingoglycolipid | 1.00 |
| 1,3-butylene glycol | 7.00 |
| Purified water | Balance |

### Formulation Example 7: Ointment

TABLE 10

| Ingredients | Content (%) |
|---|---|
| Prunus mume flower extract | 5.00 |
| Polyvinyl alcohol | 13.00 |
| L-ascorbic acid-2-phosphate | 1.00 |
| magnesium salt | |
| Lauroyl hydroxyproline | 1.00 |
| Water-soluble collagen (1% | 2.00 |
| aqueous solution) | |
| 1,3-butylene glycol | 3.00 |
| Ethanol | 5.00 |
| Purified water | Balance |

### Formulation Example 8: Preparation of Cosmetic Solution

TABLE 11

| Ingredients | Content (%) |
|---|---|
| Prunus mume flower extract | 3.00 |
| Hydroxyethylene cellulose (2% | 12.00 |
| aqueous solution) | |
| Xanthan gum (2% aqueous | 2.00 |
| solution) | |
| 1,3-butylene glycol | 6.00 |
| Concentrated glycerin | 4.00 |
| Sodium hyaluronate (1% | 2.00 |
| aqueous solution) | |
| Purified water | Balance |

### Formulation Example 9: Health Food

TABLE 12

| Ingredients | Content | |
|---|---|---|
| Prunus mume flower extract | 2 | mg |
| Vitamin A acetate | 70 | μg |
| Vitamin E | 1.0 | mg |
| Vitamin B1 | 0.13 | mg |
| Vitamin B2 | 0.15 | mg |
| Vitamin B6 | 0.5 | mg |
| Vitamin B12 | 0.2 | μg |
| Vitamin C | 10 | mg |
| Biotin | 10 | μg |
| Nicotinic acid amide | 1.7 | mg |
| Folic acid | 50 | μg |
| Calcium pantothenate | 0.5 | mg |
| Ferrous sulfate | 1.75 | mg |
| Zinc oxide | 0.82 | mg |
| Magnesium carbonate | 25.3 | mg |
| Potassium phosphate monobasic | 15 | mg |
| Calcium phosphate dibasic | 55 | mg |
| Potassium citrate | 90 | mg |
| Calcium carbonate | 100 | mg |
| Magnesium chloride | 24.8 | mg |

### Formulation Example 10: Health Beverage

TABLE 13

| Ingredients | Content | |
|---|---|---|
| Prunus mume flower extract | 50 | mg |
| Citric acid | 1000 | mg |
| Oligosaccharide | 100 | g |
| Taurine | 1 | g |

| | |
|---|---|
| Purified water | Balance |

In one aspect, it is possible to return the gene expression level which is changed by microdust to the normal level, thereby caring damages of skin cells, by using the composition for caring damages of skin cells by microdust of the present invention.

While the present invention has been described with respect to the specific embodiments, it will be apparent to those skilled in the art that various changes and modifications may be made without departing from the spirit and scope of the invention as defined in the following claims.

1. A method for caring damages of skin cells by microdust, comprising administering a composition comprising a Prunus mume flower extract as an active ingredient to a subject in need thereof.

2. The method according to claim 1,
wherein the Prunus mume flower extract is extracted with at least one extraction solvent selected from the group consisting of water, C₁-C₆ anhydrous or hydrated lower alcohol, acetone, butylene glycol, ethyl acetate, diethyl acetate, diethyl ether, benzene, chloroform and hexane.

3. The method according to claim 1,
wherein the Prunus mume flower extract is comprised from 0.000001% to 30% by weight based on a total weight of the composition.

4. The method according to claim 1,
wherein the composition inhibits the expression of IL-36G (NM_019618).

5. The method according claim 4,
wherein the composition acts on keratinocytes.

6. The method according to claim 1,
wherein a particle size of the microdust is 2.5 μm or less.

7. The method according to claim 1,
wherein the Prunus mume flower extract is administered at a dose of 10 to 500 mg/kg/day.

8. The method according to claim 1,
wherein the composition is a cosmetic composition.

9. The method according to claim 1,
wherein the composition is a pharmaceutical composition.

10. The method according to claim 1,
wherein the composition is a health functional food composition.
